SEQUENCE LISTING (27 sequences)

SEQ ID NO: 1 - protein, 86 aa, Artificial Sequence
  SYNTHETIC CONSTRUCT
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 21 aa, Artificial Sequence
  SYNTHETIC CONSTRUCT
GIVEQCCTSICSLYQLENYCN

SEQ ID NO: 3 - protein, 30 aa, Artificial Sequence
  SYNTHETIC CONSTRUCT
FVNQHLCGSHLVEALYLVCGERGFFYTPKT

SEQ ID NO: 4 - protein, 57 aa, Artificial Sequence
  SYNTHETIC CONSTRUCT
  MISC_FEATURE (16)..(16): X is Tyr or Glu
  MISC_FEATURE (28)..(28): X is is Pro or Glu
  MISC_FEATURE (29)..(29): X is Lys, Pro, or Glu
  MISC_FEATURE (32)..(32): X is Glu or Ala
  MISC_FEATURE (44)..(44): X is Thr, Glu, or His
  MISC_FEATURE (49)..(49): X is Leu,  Phe or Trp
  MISC_FEATURE (50)..(50): X is Tyr or Glu
  MISC_FEATURE (50)..(50): X is Glu, Gln, Arg, or Phe
  misc_feature (53)..(53): Xaa can be any naturally occurring amino acid
FVNQHLCGSHLVEALXLVCGERGFFYTXXTEXGPRRGIVEQCCXSICSXXQLXNYCN

SEQ ID NO: 5 - protein, 57 aa, Artificial Sequence
  SYNTHETIC CONSTRUCT
FVNQHLCGSHLVEALYLVCGERGFFYTPETEAGPRRGIVEQCCESICSLEQLQNYCN

SEQ ID NO: 6 - protein, 57 aa, Artificial Sequence
  SYNTHETIC CONSTRUCT
FVNQHLCGSHLVEALYLVCGERGFFYTPETEAGPRRGIVEQCCESICSLEQLRNYCN

SEQ ID NO: 7 - protein, 57 aa, Artificial Sequence
  SYNTHETIC CONSTRUCT
FVNQHLCGSHLVEALYLVCGERGFFYTPETEAGPRRGIVEQCCHSICSLEQLQNYCN

SEQ ID NO: 8 - protein, 57 aa, Artificial Sequence
  SYNTHETIC CONSTRUCT
FVNQHLCGSHLVEALYLVCGERGFFYTEPTEAGPRRGIVEQCCESICSLEQLRNYCN

SEQ ID NO: 9 - protein, 57 aa, Artificial Sequence
  SYNTHETIC CONSTRUCT
FVNQHLCGSHLVEALYEVCGERGFFYTPETEEGPRRGIVEQCCHSICSLEQLENYCN

SEQ ID NO: 10 - protein, 57 aa, Artificial Sequence
  SYNTHETIC CONSTRUCT
FVNQHLCGSHLVEALYFVCGERGFFYTPETEEGPRRGIVEQCCHSICSLEQLENYCN

SEQ ID NO: 11 - protein, 57 aa, Artificial Sequence
  SYNTHETIC CONSTRUCT
FVNQHLCGSHLVEALYLVCGERGFFYTPETEEGPRRGIVEQCCHSICSFEQLENYCN

SEQ ID NO: 12 - protein, 57 aa, Artificial Sequence
  SYNTHETIC CONSTRUCT
FVNQHLCGSHLVEALELVCGERGFFYTPETEEGPRRGIVEQCCHSICSLEQEENYCN

SEQ ID NO: 13 - protein, 57 aa, Artificial Sequence
  SYNTHETIC CONSTRUCT
FVNQHLCGSHLVEALYLVCGERGFFYTPETEAGPRRGIVEQCCHSICSLEQLRNYCN

SEQ ID NO: 14 - protein, 57 aa, Artificial Sequence
  SYNTHETIC CONSTRUCT
FVNQHLCGSHLVEALYLVCGERGFFYTPETEAGPRRGIVEQCCHSICSWEQLQNYCN

SEQ ID NO: 15 - protein, 57 aa, Artificial Sequence
  SYNTHETIC CONSTRUCT
FVNQHLCGSHLVEALYLVCGERGFFYTEPTEAGPRRGIVEQCCESICSLEQLQNYCN

SEQ ID NO: 16 - protein, 57 aa, Artificial Sequence
  SYNTHETIC CONSTRUCT
FVNQHLCGSHLVEALYLVCGERGFFYTEPTEAGPRRGIVEQCCHSICSLEQLQNYCN

SEQ ID NO: 17 - protein, 57 aa, Artificial Sequence
  SYNTHETIC CONSTRUCT
FVNQHLCGSHLVEALYLVCGERGFFYTEPTEAGPRRGIVEQCCHSICSLEQLRNYCN

SEQ ID NO: 18 - protein, 57 aa, Artificial Sequence
  SYNTHETIC CONSTRUCT
FVNQHLCGSHLVEALYLVCGERGFFYTEPTEAGPRRGIVEQCCHSICSWEQLENYCN

SEQ ID NO: 19 - protein, 57 aa, Artificial Sequence
  SYNTHETIC CONSTRUCT
FVNQHLCGSHLVEALYLVCGERGFFYTPETEAGPRRGIVEQCCESICSLLQLQNYCN

SEQ ID NO: 20 - protein, 57 aa, Artificial Sequence
  SYNTHETIC CONSTRUCT
FVNCHLCGSHLVEALYLVCGECGFFYTPETEEGPAHGIVEQCCHSCCSLEQLENYCN

SEQ ID NO: 21 - protein, 57 aa, Artificial Sequence
  SYNTHETIC CONSTRUCT
FVNCHLCGSHLVEALYLVCGEAGFFYTPETEEGPAHGIVEQCCHSCCSLEQLENYCN

SEQ ID NO: 22 - protein, 30 aa, Artificial Sequence
  SYNTHETIC CONSTRUCT
FVNQHLCGSHLVEALYLVCGERGFFYTKPT

SEQ ID NO: 23 - protein, 30 aa, Artificial Sequence
  SYNTHETIC CONSTRUCT
FVNQHLCGSDLVEALYLVCGERGFFYTPKT

SEQ ID NO: 24 - protein, 30 aa, Artificial Sequence
  SYNTHETIC CONSTRUCT
FVNQHLCGSDLVEALYLVCGERGFFYTKPT

SEQ ID NO: 25 - protein, 57 aa, Artificial Sequence
  SYNTHETIC CONSTRUCT
FVNQHLCGSHLVEALYLVCGERGFFYTPETEEGPRRGIVEQCCESICSLEQLRNYCN

SEQ ID NO: 26 - protein, 57 aa, Artificial Sequence
  SYNTHETIC CONSTRUCT
FVNQHLCGSHLVEALYLVCGERGFFYTPETEEGPRRGIVEQCCHSICSLEQLRNYCN

SEQ ID NO: 27 - protein, 57 aa, Artificial Sequence
  SYNTHETIC CONSTRUCT
FVNQHLCGSHLVEALYNVCGERGFFYTPETEEGPRRGIVEQCCHSICSLEQLENYCN

(19) United States
(12) Patent Application Publication
(10) Pub. No.: US 2021/0371489 A1
(43) Pub. Date: Dec. 2, 2021

(54) SITE 2 SINGLE-CHAIN INSULIN ANALOGUES

(71) Applicant: Case Western Reserve University, Cleveland, OH (US)

(72) Inventor: Michael A. WEISS, Indianapolis, IN (US)

(21) Appl. No.: 17/277,979

(22) PCT Filed: Sep. 23, 2019
(86) PCT No.: PCT/US2019/052345

§ 371 Date: Mar. 19, 2021

Related U.S. Application Data
(60) Provisional application No. 62/765,960, filed on Sep. 21, 2018.

(51) Int. Cl. C07K 14/62

(52) U.S. Cl. CPC C07K 14/62; A61K 38/00

(57) ABSTRACT

A single-chain insulin analogues may comprise an insulin B-chain polypeptide sequence connected by a connecting polypeptide (or C-domain) sequence to an insulin A-chain polypeptide sequence. The connecting polypeptide sequence may be Glu-Xaa-Gly-Pro-Arg-Arg where Xaa is Glu or Ala. The insulin analogues may additionally comprise Glu or His substitutions at the position corresponding to A8 of human insulin and/or a Glu substitution at the position corresponding to A14 of human insulin. In some embodiments, the insulin analogues may additionally comprise either a Pro or Glu at the positions corresponding to B28 and B29 of wild-type insulin. Additional substitutions may comprise Phe or Trp at the position corresponding to A13 of wild type insulin and/or Gln, Arg, Phe, or Glu at the position corresponding to A17 of wild type insulin. In some embodiments, a Glu substitution at the position corresponding to B16 of wild type insulin may be present. In other embodiments, a Cys substitution may be present at the positions corresponding to A10 and/or B4 of wild-type insulin. In addition or in the alternative, the analogue may comprise a His or Ala substitution at the position corresponding to B22 of wild-type insulin and/or the connecting polypeptide sequence may be Glu-Glu-Gly-Pro-Ala-His. A method of treating a patient with diabetes mellitus comprises administering a physiologically effective amount of the insulin analogue or a physiologically acceptable salt thereof to a patient by means of intravenous, intraperitoneal, or subcutaneous injection.

### CROSS REFERENCE TO RELATED APPLICATIONS

This application is a national stage application of PCT/US2019/052345, filed on Sep. 23, 2019, which claims benefit of U.S. Provisional Application No. 62/765,960, filed on Sep. 21, 2018. The disclosures of the above-referenced applications are incorporated herein by reference.

### STATEMENT REGARDING FEDERALLY SPONSORED RESEARCH OR DEVELOPMENT

This invention was made with government support under grant numbers DK040949 and DK074176 awarded by the National Institutes of Health. The government has certain rights in the invention.

### BACKGROUND OF THE INVENTION

This invention relates to polypeptide hormone analogues that exhibits enhanced pharmaceutical properties, such as altered pharmacokinetic and pharmacodynamic properties, i.e., conferring foreshortened duration of action relative to soluble formulations of the corresponding wild-type human hormone. More particularly, this invention relates to single-chain insulin analogues exhibiting such properties. Even more particularly, this invention relates to insulin analogues containing (i) one or more amino-acid substitutions in its “Site-2 receptor-binding surface” in conjunction optionally with (ii) one or more B-chain substitutions known in the art to accelerate the absorption of an insulin analogue from a subcutaneous depot into the blood stream. The insulins analogues of the present invention contain a connecting domain (C domain) between A- and B-chains (and so be described as single-chain analogues) and may optionally contain standard or non-standard amino-acid substitutions at other sites in the A- or B chains.

The essential idea underlying the present invention is to enhance the safety and efficacy of rapid-acting analogues through the simultaneous incorporation of substitutions in the Site-2 receptor-binding surface of the hormone. This combination of substitutions confers “fast-on/fast-off” pharmacokinetic properties of utility in the prandial control of blood glucose concentration following subcutaneous injection as a method of treatment of diabetes mellitus and of further utility in the algorithm-based operation of closed-loop systems for the treatment of diabetes mellitus (“smart pumps”).

The engineering of proteins, including therapeutic agents and vaccines, may have broad medical and societal benefits. Naturally occurring proteins—as encoded in the genomes of human beings, other mammals, vertebrate organisms, invertebrate organisms, or eukaryotic cells in general—often contain two or more functional surfaces. A benefit of protein analogues would be to achieve selective modification of one or the other of these functional surfaces, such as to provide fine-tuning of biological activity. An example of a therapeutic protein is provided by insulin. The three-dimensional structure of wild-type insulin has been well characterized as a zinc hexamer, as a zinc-free dimer, and as an isolated monomer in solution (FIGS. 1 and 2). Wild-type human insulin and insulin molecules encoded in the genomes of other mammals bind to insulin receptors (IRs), each of which containing multiple domains and associated domain surfaces. The IR is a dimer of αβ half-receptors (designated (αβ)₂) wherein the α chain and β chain are the post-translational products of a single precursor polypeptide. The hormone-binding surfaces of the (αβ)₂ dimer has been classified as Site 1 and Site 2 in relation to the non-linear binding and kinetic properties of the receptor. This binding scheme is shown in schematic form in FIG. 3. Recent advances in structural and biochemical analysis of fragments of the IR ectodomain have shown that Site 1 consists of a trans-binding element formed by both a subunits in the (αβ)₂ dimer: the N-terminal L1 domain of one subunit and the C-terminal α-helix (αCT) of the other. The location of Site 2 is not well characterized but is proposed to comprise parts of the first and second fibronectin-homology domains.

The receptor-binding surfaces of insulin or insulin analogues may likewise be classified on a cognate basis: the respective Site-1-binding surface (classical receptor-binding surface) and Site 2-binding surface (non-classical receptor-binding surface). The Site-1-binding surface of insulin overlaps its dimer-forming interface in the B chain whereas the Site-2-binding surface overlaps its hexamer-forming interface. The Site 1 hormone-IR interface has recently been visualized at low resolution. Presumptive Site 2-related residues may be defined either based on kinetic effects of mutations or based on positions that are extrinsic to site 1 wherein mutations nonetheless impair binding. These criteria highlight the potential importance of non-classical residues A12, A13, A17, B13 and B17. Respective Site-1-related and Site-2-related surfaces are shown in relation to the surface of an insulin monomer in FIG. 4. Whereas substitutions known in the art to accelerate the absorption of insulin from a subcutaneous depot are ordinarily within and adjacent to the Site-1-binding surface of the hormone (such as at residues B24, B28 or B29), we envisaged that modification of the Site-2-binding surface could modulate the cellular duration of signaling by the hormone-receptor complex once engaged at the surface of a target cell or tissue. Although not wishing to be restricted by theory, we further envisioned that such foreshortening of the cellular duration of signaling would confer “fast-off” pharmacokinetic properties to a rapid-acting insulin analogue (“fast-on”) whose disassembly in the subcutaneous depot had been accelerated by substitutions within or adjacent to the Site-1-binding surface as known in the art (see FIG. 6 for rationale). Thus, such a novel combination of Site-1/Site-2-related substitutions would together confer the desirable combination of fast-on/fast-off pharmacokinetic properties of novel utility in the treatment of diabetes mellitus.

It is known in the art that modifications or substitutions within the classical receptor-binding surface of insulin may impair the in vitro affinity of the hormone for its receptor by up to ca. fivefold (e.g., from a dissociation constant of 0.05 nM to a dissociation constant of 0.25 nM) without significant effect on in vivo potency as assessment by the ability of the variant insulin, when injected subcutaneously or intravenously, to cause a reduction in blood glucose concentration. Such robustness is, at least in part, attributed to a compensating relationship between affinity and rate of clearance of the hormone from the bloodstream. Because binding to the IR mediates both insulin action and, to a large extent, insulin clearance, a reduction in affinity leads to a proportionate increase in the circulatory half-life and hence opportunity to effect biological signaling. Examples of such compensation have been disclosed in relation to insulin analogues in which the Phenylalanine at position B24 is substituted by Cyclohexanylalanine (Cha), disclosed in U.S. Pat. No. 9,487,572, issued Nov. 8, 2016, the disclosure of which are incorporated by reference herein. The non-planar aliphatic ring of Cha at position B24 (illustrated in FIG. 8) impairs receptor-binding affinity by ca. threefold but has no effect on the potency or pharmacodynamics properties of KP-insulin as tested in diabetic Sprague-Dawley rats (FIG. 9A). Additional examples have been provided by modified by fluoro-aromatic and chloro-aromatic substitutions at position B24 as illustrated in FIG. 7 (in relation to the wild-type dimer interface) and as disclosed in U.S. Pat. No. 9,079,975, issued Jul. 14, 2015, U.S. Pat. No. 8,921,313 issued Dec. 30, 2014, and U.S. Pat. No. 9,908,925 issued Mar. 6, 2018, which are incorporated by reference herein.

Modification of Phe^{B24} in insulin Lispro ([LysB28, ProB29]-insulin, also designated KP-insulin (the active component of Humalog®); Eli Lilly and Co., Indianapolis, Ind.) by a single chloro-substitution at the ortho position of the aromatic ring (2-Cl-PheB24) thus impairs the biochemical affinity of the variant hormone for the isolated IR in vitro by ca. threefold without change in its in vivo potency as assessed in Sprague-Dawley rats rendered diabetic by streptozotocin or as assessed in anesthetized non-diabetic adolescent Yorkshire pigs in which endogenous insulin secretion was suppressed by intravenous octreotide (data not shown). Even more dramatically, whereas a derivative of [AspB10, LysB28, ProB29]-insulin (DKP-insulin) containing 2-F-PheB24 (ortho-monofluoro-phenylalanine at position B24) exhibits a similar decrement in receptor-binding affinity (ca. 35(±5)% relative to KP-insulin), its potency and duration of signaling are enhanced in diabetic Sprague-Dawley rats (FIG. 9B) as disclosed in in U.S. Pat. No. 8,921,313, referenced above. It would be expected, therefore, that modifications or substitutions generally in the insulin molecule that introduce moderate perturbations to IR binding as evaluated in vitro would be well tolerated in vivo and be indistinguishable from wild-type insulin with respect to potency or (in the absence of effects on self-assembly) other pharmacological properties—or even more potent and prolonged as in the case of the 2-F-PheB24-DKP-insulin. Although side chains at positions B13, B17, A12, A13, and A17 are not thought to be engaged at the primary hormone-binding surface of the insulin receptor, alanine scanning mutagenesis has shown that single Alanine substitutions at Site-2-related positions affect relative receptor-binding affinities as follows: (position B13) 12(±3)%, (B17) 62(±14)%, (A12) 108(±28)%, (A13) 30(±7)%, and (A17) 56(±20)%.

Administration of insulin has long been established as a treatment for diabetes mellitus. A major goal of conventional insulin replacement therapy in patients with diabetes mellitus is tight control of the blood glucose concentration to prevent its excursion above or below the normal range characteristic of healthy human subjects. Excursions below the normal range are associated with immediate adrenergic or neuroglycopenic symptoms, which in severe episodes lead to convulsions, coma, and death. Excursions above the normal range are associated with increased long-term risk of microvascular disease, including retinapathy, blindness, and renal failure. Insulin is a small globular protein that plays a central role in metabolism in vertebrates. Insulin contains two chains, an A chain, containing 21 residues, and a B chain containing 30 residues; individual residues are indicated by the identity of the amino acid (typically using a standard three-letter code), the chain and sequence position (typically as a superscript). The hormone is stored in the pancreatic β-cell as a Zn²⁺-stabilized hexamer, but functions as a Zn²⁺-free monomer in the bloodstream. Insulin is the product of a single-chain precursor, proinsulin, in which a connecting region (35 residues) links the C-terminal residue of B chain (residue B30) to the N-terminal residue of the A chain. A variety of evidence indicates that it consists of an insulin-like core and disordered connecting peptide. Formation of three specific disulfide bridges (A6-A11, A7-B7, and A20-B19; labeled in FIG. 2) is thought to be coupled to oxidative folding of proinsulin in the rough endoplasmic reticulum (ER). Proinsulin assembles to form soluble Zn²⁺-coordinated hexamers shortly after export from ER to the Golgi apparatus. Endoproteolytic digestion and conversion to insulin occurs in immature secretory granules followed by morphological condensation. Crystalline arrays of zinc insulin hexamers within mature storage granules have been visualized by electron microscopy (EM).

There is a medical and societal need to engineer a rapid-acting two-chain insulin analogue or single-chain insulin analogue that combines (i) accelerated disassembly of an insulin complex in the subcutaneous depot with (ii) foreshortened duration of cellular signaling once the hormone-receptor complex is engaged at the surface of target cells or tissues. There is also an unmet need of a subset of patients treated with prandial insulin injections to avoid late post-prandial hypoglycemia and the unmet performance specifications of closed-loop algorithm-based pump systems (“smart pumps”) with respect to safety and efficacy. Feedback control in a smart pump would be made more robust by a foreshortened duration of signaling as effects of over-delivery events would be curtailed. It would be desirable, therefore, to provide a novel class of insulin analogues that combined modifications in the B chain designed to accelerate disassembly of an insulin complex with modifications elsewhere in the protein molecule that lead to foreshortened duration of signaling.

While engineered proteins can provide benefits such as selective activity and duration of action, undesirable effects can also arise, such as binding to homologous cellular receptors associated with promotion of the growth of cancer cells. Wild-type insulin binds not only to insulin receptor but also binds with lower affinity to the homologous Type 1 insulin-like growth factor receptor (IGF-1R). Certain alterations, like those associated with Asp^{B10} and other substitutions at position B10, have elicited broad concern due to possible association with an increased risk of cancer in animals or human patients taking such analogues. This concern is especially marked with respect to basal insulin analogues, i.e., those designed for once-a-day administration with 12-24 hour profile of insulin absorption from a subcutaneous depot and 12-24 hour profile of insulin action. This includes prior single-chain insulin analogues such as those disclosed in U.S. Pat. Pub. No. US 2011/0195896, entitled “Isoform-Specific Insulin Analogues,” published Aug. 11, 2011 (and incorporated by reference herein). A single-chain insulin analogue with high receptor-binding affinity was described therein which the foreshortened C-domain was the 12-residue C-domain of insulin-like growth factor I (IGF-I; sequence GYGSSSRRAPQT; SEQ ID NO: 12), yielding a chimeric protein. However, such chimeric molecules exhibit enhanced relative and absolute affinities for IGF-1R. There is a need therefore, for a single-chain insulin analogue with decreased cross-affinity for IGF-1R.

### SUMMARY OF THE INVENTION

It is, therefore, an aspect of the present invention to provide single-chain insulin analogues that provide (i) rapid absorption into the blood stream due to substitutions or modifications in or adjoining the Site-1-related surface of the B chain and (ii) foreshortened duration of target cell signaling due to mutations or modifications of the Site-2-related surface of the A- and/or B chain. The analogues of the present invention contain at least a portion of the biological activity of wild-type insulin to direct a reduction in the blood glucose concentration on subcutaneous or intravenous injection. It is an aspect of the present invention that the isoelectric points of the analogues lie in the range 3.5-6.0 such that formulation as a clear soluble solution in the pH range 6.8-8.0 is feasible.

The single-chain insulin analogues of the present invention may contain Histidine at position B10 and so be amenable to formulation as zinc insulin hexamers. Optionally, the analogues of the present invention may contain Aspartic Acid at position B10 when combined with a substitution or modification elsewhere in the protein such that the analogue exhibits an affinity for the IR is equal to or less than that of wild-type insulin (and so unlikely to exhibit prolonged residence times in the hormone-receptor complex) and an affinity for the Type 1 IGF-1 receptor is equal to or less than that of wild-type insulin (and so unlikely to exhibit IGF-I-related mitogenicity).

In general, the single-chain insulin analogues of the present invention may comprise an insulin B-chain polypeptide sequence connected by a connecting polypeptide (or C-domain) sequence to an insulin A-chain polypeptide sequence. The connecting polypeptide sequence may be Glu-Xaa-Gly-Pro-Arg-Arg where Xaa is Glu or Ala. The insulin analogues may additionally comprise Glu or His substitutions at the position corresponding to A8 of human insulin and/or a Glu substitution at the position corresponding to A14 of human insulin. In some embodiments, the insulin analogues may additionally comprise either a Pro or Glu at the positions corresponding to B28 and B29 of wild-type insulin. Additional substitutions may comprise Phe or Trp at the position corresponding to A13 of wild type insulin and/or Gln, Arg, Phe, or Glu at the position corresponding to A17 of wild type insulin. In some embodiments, a Glu substitution at the position corresponding to B16 of wild type insulin may be present. In other embodiments, a Cys substitution may be present at the positions corresponding to A10 and/or B4 of wild-type insulin. In addition or in the alternative, the analogue may comprise a His or Ala substitution at the position corresponding to B22 of wild-type insulin and/or the connecting polypeptide sequence may be Glu-Glu-Gly-Pro-Ala-His.

Pertinent to the present invention is the invention of novel foreshortened C domains of length 6-11 residues in place of the 36-residue wild-type C domain characteristic of human proinsulin. Single-chain insulin analogues provide a favorable approach toward the design of fibrillation-resistant insulin analogues amenable to formulation as zinc-free monomers. Such single-chain analogues may be designed to bear substitutions within or adjoining the Site-1-binding surface of the B chain such as to confer rapid-acting pharmacokinetics. Single-chain insulin analogues suitable to further modification at one or more positions selected from B13, B17, A12, A13, or A17 are as disclosed in U.S. Pat. Pub. No. US2011/0195896 (filed Oct. 22, 2010) and U.S. Pat. No. 8,192,957, which are incorporated by reference herein.

It was surprisingly discovered that substitutions or modifications within Site 2 can markedly foreshorten the duration of insulin action in vivo (an alteration of pharmacodynamics) despite conferring perturbations to the biochemical affinity of the variant insulin for the IR of less than fivefold.

### BRIEF DESCRIPTION OF THE SEVERAL VIEWS OF THE DRAWINGS

FIG. 1 is a representation of the structure of insulin in a typical pharmaceutical formulation and as an isolated monomer in the bloodstream. (A) The phenol-stabilized R₆ zinc hexamer. Axial zinc ions (overlaid) are shown as coincident black spheres coordinated by histidine side chains. The A-chain is shown in dark gray, and B-chain in medium gray (residues B1-B8) and light gray (B9-B30). (B) Structure of an insulin monomer. The A chain is shown in dark gray, and B chain in medium gray; disulfide brides are depicted as balls and sticks (labels are provided in FIG. 2).

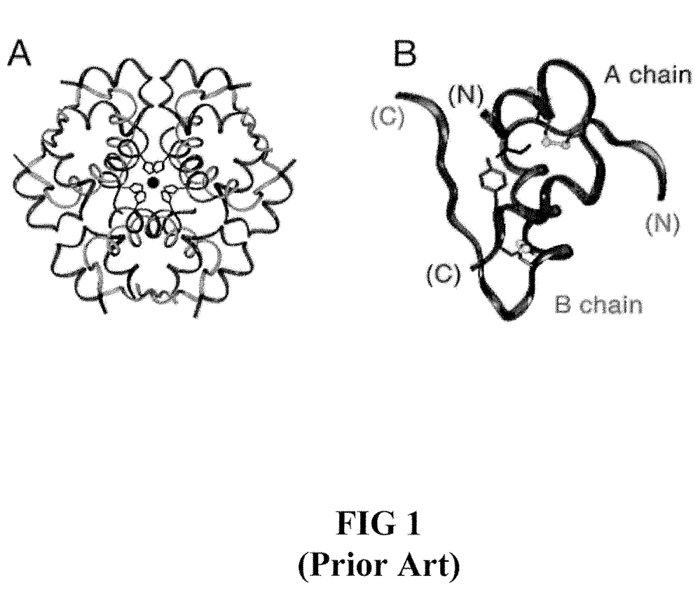

FIG. 2 is a representation of the structure of insulin dimer and core Beta-sheet. Residues B24-B28 (medium gray) for an anti-parallel Beta-sheet, repeated three times in the hexamer by symmetry. The A- and B chains are otherwise shown in light and dark gray, respectively. The position of PheB24 is highlighted in the arrow in dark gray. Cystines are identified by sulfur atoms that are shown as spheres. Coordinates were obtained from T6 hexamer (PDB 4INS).

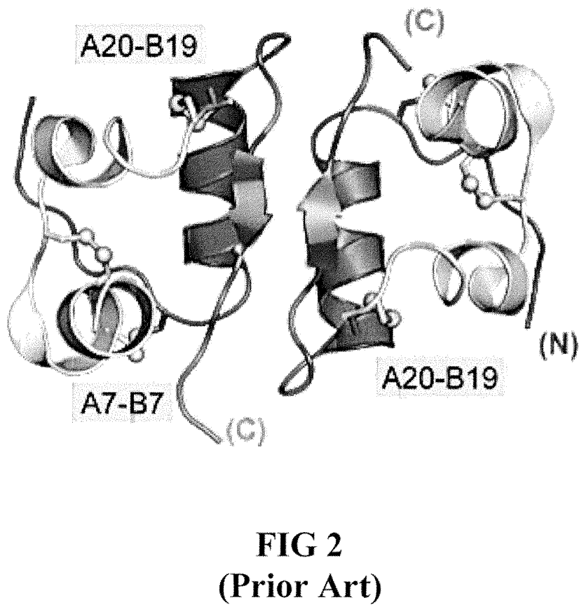

FIG. 3 is a representation of a model of the insulin receptor: each α subunit of the receptor contains two distinct insulin-binding sites: Site 1 (high affinity) and Site 2 (low affinity but critical to signal propagation). Specific insulin binding bridges the two α subunits, in turn altering the orientation between β subunits, communicating a signal to the intracellular tyrosine kinase (TK) domain.

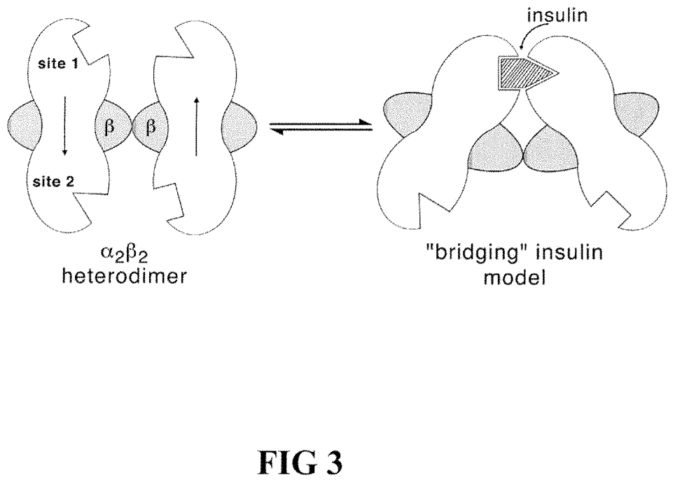

FIG. 4 is a representation of the functional surfaces of insulin. Whereas the classical receptor-binding surface of insulin engages IR Site 1 (B12, B16, B24-B26), its Site 2-related surface includes hexamer contacts Val^{B17} and Leu^{A13}; proposed Site 2 residues are shown (B13, B17, A12, A13, and A17) with addition of neighboring residue B10, which may contribute to both Sites 1 and 2. The A- and B chains are otherwise shown in light gray and dark gray, respectively.

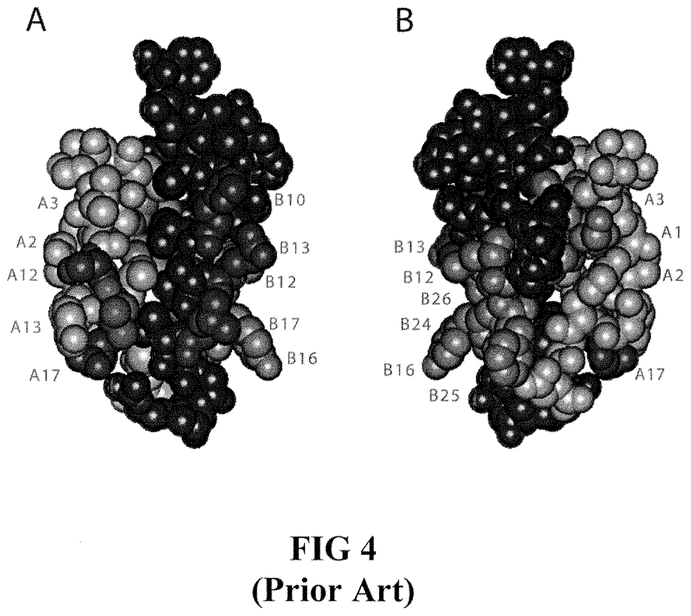

FIG. 5 is a representation of the position of Leu^{A13} on the surface of an insulin hexamer, dimer and monomer. Coordinates were obtained from R₆ hexamer (PDB 1TRZ).

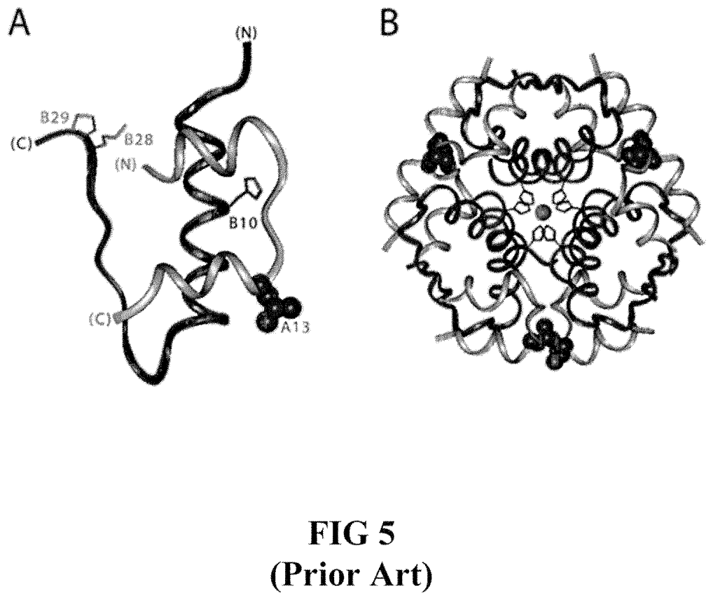

FIG. 6 is a representation of the rationale for the design and formulation of mealtime insulin analogues. Rapid dissociation of the zinc hexamer yields dimers and monomers able to enter the capillaries. Current mealtime insulin analogs contain standard substitutions at the edge of the core Beta-sheet.

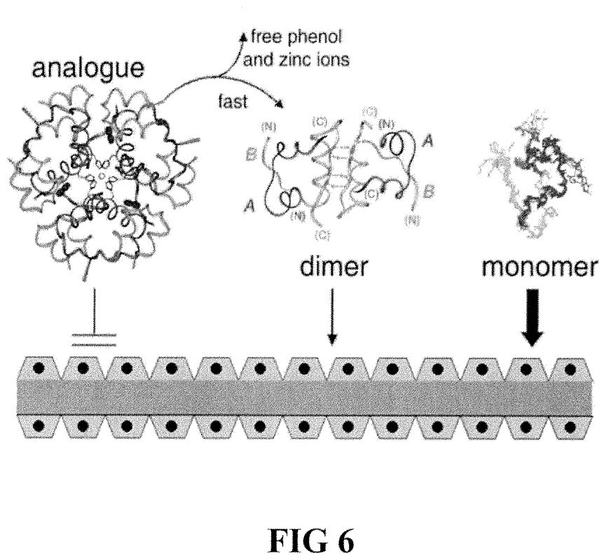

FIG. 7 is a bar graph showing the decrease in blood sugar levels in male Lewis rats rendered diabetic by treatment with streptozotocin after treatment with streptozotocin after treatment with subcutaneously injected single-chain insulin analogues of the present invention.

FIG. 8 is a bar graph showing the number of days for fibril formation for insulin lispro and single-chain insulin analogues of the claimed invention.

FIG. 9 is a bar graph showing the binding of single-chain insulin analogues of the claimed invention with human type 1 insulin-like growth factor receptor relative to insulin lispro.

FIG. 10 is a bar graph showing the fold change in the ratio of Cyclin D1:Cyclin G2 expression following treatment with the single-chain insulin analogues of the present invention.

FIG. 11 is a bar graph showing the calculated free energy of unfolding (AGO of single-chain insulin analogues of the present invention and human insulin (HI), insulin lispro (KP) and DB 10 KP insulin.

### DETAILED DESCRIPTION OF THE INVENTION

The present invention is directed toward a single-chain insulin analogue that may provide an extended fibrillation time and decreased affinity for human type 1 insulin-like growth factor receptor (hIGFR) compared to insulin lispro, while retaining at least a portion of the blood sugar glucose-lowering activity compared to insulin lispro. The single-chain insulins may also provide decreased mitogenicity compared to human insulin and/or an insulin analogue containing and Asp (or D) substitution at position B10.

In general, the single-chain insulin analogues of the present invention comprise constitute an insulin B-chain polypeptide sequence connected by a connecting polypeptide (or C-domain) sequence to an insulin A-chain polypeptide sequence. The connecting polypeptide sequence may be Glu-Xaa-Gly-Pro-Arg-Arg (EXGPRR) where Xaa (X) is Glu (E) or Ala (A). The insulin analogues may additionally comprise Glu (E) or His (H) substitutions at the position corresponding to A8 of human insulin and/or a Glu (E) substitution at the position corresponding to A14 of human insulin. In some embodiments, the insulin analogues may additionally comprise either a Pro (P) or Glu (E) at the positions corresponding to B28 and B29 of wild-type insulin. Additional substitutions may comprise Phe (F) or Trp (W) at the position corresponding to A13 of wild type insulin and/or Gln (Q), Arg (R), Phe (F), or Glu (E) at the position corresponding to A17 of wild type insulin. In some embodiments, a Glu substitution at the position corresponding to B16 of wild type insulin may be present. In other embodiments, a Cys (C) substitution may be present at the positions corresponding to A10 and/or B4 of wild-type insulin. In addition or in the alternative, the analogue may comprise a His (H) or Ala (A) substitution at the position corresponding to B22 of wild-type insulin and/or the connecting polypeptide sequence may be Glu-Glu-Gly-Pro-Ala-His (EEGPAH).

In some embodiments, it is a feature of the present invention that the isoelectric point of the single-chain analogue is between 3.5 and 6.0 such that a soluble formulation neutral conditions (pH 6.8-8.0) would be feasible.

It is also envisioned that single-chain analogues may also be made with A- and B-domain sequences derived from animal insulins, such as porcine, bovine, equine, and canine insulins, by way of non-limiting examples. In addition or in the alternative, the insulin analogue of the present invention may contain a deletion of residues B1-B3 or may be combined with a variant B chain lacking Lysine (e.g., LysB29 in wild-type human insulin) to avoid Lys-directed proteolysis of a precursor polypeptide in yeast biosynthesis in Pichia pastoris, Saccharomyces cerevisiae, or other yeast expression species or strains. The B-domain of the single-chain insulin of the present invention may optionally contain non-standard substitutions, such as D-amino-acids at positions B20 and/or B23 (intended to augment thermodynamic stability, receptor-binding affinity, and resistance to fibrillation), a halogen modification at the 2 ring position of PheB24 (i.e., ortho-F-PheB24, ortho-Cl-PheB24, or ortho-Br-PheB24; intended to enhance thermodynamic stability and resistance to fibrillation), 2-methyl ring modification of PheB24 (intended to enhance receptor-binding affinity). It is also envisioned that ThrB27, ThrB30, or one or more Serine residues in the C-domain may be modified, singly or in combination, by a monosaccaride adduct; examples are provided by O-linked N-acetyl-β-D-galactopyranoside (designated GalNAc-O^{β}-Ser or GalNAc-O^{β}-Thr), O-linked α-D-mannopyranoside (mannose-O^{β}-Ser or mannose-O^{β}-Thr), and/or α-D-glucopyranoside (glucose-O^{β}-Ser or glucose-O^{β}-Thr).

Furthermore, in view of the similarity between human and animal insulins, and use in the past of animal insulins in human patients with diabetes mellitus, it is also envisioned that other minor modifications in the sequence of insulin may be introduced, especially those substitutions considered “conservative.” For example, additional substitutions of amino acids may be made within groups of amino acids with similar side chains, without departing from the present invention. These include the neutral hydrophobic amino acids: Alanine (Ala or A), Valine (Val or V), Leucine (Leu or L), Isoleucine (Ile or I), Proline (Pro or P), Tryptophan (Trp or W), Phenylalanine (Phe or F) and Methionine (Met or M). Likewise, the neutral polar amino acids may be substituted for each other within their group of Glycine (Gly or G), Serine (Ser or S), Threonine (Thr or T), Tyrosine (Tyr or Y), Cysteine (Cys or C), Glutamine (Glu or Q), and Asparagine (Asn or N). Basic amino acids are considered to include Lysine (Lys or K), Arginine (Arg or R) and Histidine (His or H). Acidic amino acids are Aspartic acid (Asp or D) and Glutamic acid (Glu or E). Unless noted otherwise or wherever obvious from the context, the amino acids noted herein should be considered to be L-amino acids. Standard amino acids may also be substituted by non-standard amino acids belong to the same chemical class. By way of non-limiting example, the basic side chain Lys may be replaced by basic amino acids of shorter side-chain length (Ornithine, Diaminobutyric acid, or Diaminopropionic acid). Lys may also be replaced by the neutral aliphatic isostere Norleucine (Nle), which may in turn be substituted by analogues containing shorter aliphatic side chains (Aminobutyric acid or Aminopropionic acid).

The amino-acid sequence of human proinsulin is provided, for comparative purposes, as SEQ ID NO: 1.

(human proinsulin)
SEQ ID NO: 1
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr-Arg-Arg-Glu-Ala-Glu-Asp-
Leu-Gln-Val-Gly-Gln-Val-Glu-Leu-Gly-Gly-Gly-Pro-
Gly-Ala-Gly-Ser-Leu-Gln-Pro-Leu-Ala-Leu-Glu-Gly-
Ser-Leu-Gln-Lys-Arg-Gly-Ile-Val-Glu-Gln-Cys-Cys-
Thr-Ser-Ile-Cys-Ser-Leu-Tyr-Gln-Leu-Glu-Asn-Tyr-
Cys-Asn

The amino-acid sequence of the A-chain of human insulin is provided as SEQ ID NO: 2.

(human A chain)
SEQ ID NO: 2
Gly-Ile-Val-Glu-Gln-Cys-Cys-Thr-Ser-Ile-Cys-Ser-
Leu-Tyr-Gln-Leu-Glu-Asn-Tyr-Cys-Asn

The amino-acid sequence of the B chain of human insulin is provided as SEQ ID NO: 3.

(human B chain)
SEQ ID NO: 3
Phe-Val-Asn-Gln-His-Leu-Cys-Gly-Ser-His-
Leu-Val-Glu-Ala-Leu-Tyr-Leu-Val-Cys-Gly-
Glu-Arg-Gly-Phe-Phe-Tyr-Thr-Pro-Lys-Thr

Some embodiments of the single-chain insulin analogues of the present invention may be summarized with reference to SEQ ID NO: 4.

SEQ ID NO: 4
Phe-Val-Asn-Gln-His-Leu-Cys-Gly-Ser-His-
Leu-Val-Glu-Ala-Leu-Xaa-Leu-Val-Cys-Gly-
Glu-Arg-Gly-Phe-Phe-Tyr-Thr-Xaa-Xaa-Thr-
Glu-Xaa-Gly-Pro-Arg-Arg-Gly-Ile-Val-Glu-
Gln-Cys-Cys-Xaa-Ser-Ile-Cys-Ser-Xaa-Xaa-
Gln-Leu-Xaa-Asn-Tyr-Cys-Asn,

where Xaa at position 16 (corresponding to position B16 relative to wild type insulin) is Tyr (as in wild type insulin) or Glu; Xaa at position 28 (corresponding to position B28 relative to wild type insulin) is Pro (as in wild type insulin) or Glu; Xaa at position 29 (corresponding to position B29 relative to wild type insulin) is Lys (as in wild type insulin), Pro, or Glu; Xaa at position 32 (corresponding to the second amino acid of the linker sequence between the B- and A-chains of insulin) is Glu or Ala; Xaa at position 44 (corresponding to position A8 relative to wild type insulin) is Thr (as in wild type insulin), Glu, or His; Xaa at position 49 (corresponding to position A13 relative to wild type insulin) is Leu (as in wild type insulin), Phe or Trp; Xaa at position 50 (corresponding to position A14 relative to wild type insulin) is Tyr (as in wild type insulin) or Glu; and Xaa at position 53 (corresponding to position A17 relative to wild type insulin) is Glu (as in wild type insulin), Gln, Arg, or Phe.

In one embodiment, the amino-acid sequence of the single-chain insulin, designated EA8, EA14, QA17, PE, EAGPRR, is provided as SEQ ID NO: 5.

SEQ ID NO: 5
Phe-Val-Asn-Gln-His-Leu-Cys-Gly-Ser-His-
Leu-Val-Glu-Ala-Leu-Tyr-Leu-Val-Cys-Gly-
Glu-Arg-Gly-Phe-Phe-Tyr-Thr-Pro-Glu-Thr-
Glu-Ala-Gly-Pro-Arg-Arg-Gly-Ile-Val-Glu-
Gln-Cys-Cys-Glu-Ser-Ile-Cys-Ser-Leu-Glu-
Gln-Leu-Gln-Asn-Tyr-Cys-Asn.

In another embodiment, the amino-acid sequence of the single-chain insulin, designated EA8, EA14, RA17, PE, EAGPRR, is provided as SEQ ID NO: 6.

SEQ ID NO: 6
Phe-Val-Asn-Gln-His-Leu-Cys-Gly-Ser-His-
Leu-Val-Glu-Ala-Leu-Tyr-Leu-Val-Cys-Gly-
Glu-Arg-Gly-Phe-Phe-Tyr-Thr-Pro-Glu-Thr-
Glu-Ala-Gly-Pro-Arg-Arg-Gly-Ile-Val-Glu-
Gln-Cys-Cys-Glu-Ser-Ile-Cys-Ser-Leu-Glu-
Gln-Leu-Arg-Asn-Tyr-Cys-Asn.

In another embodiment, the amino-acid sequence of the single-chain insulin, designated HA8, EA14, QA17, PE, EAGPRR, is provided as SEQ ID NO: 7.

SEQ ID NO: 7
Phe-Val-Asn-Gln-His-Leu-Cys-Gly-Ser-His-
Leu-Val-Glu-Ala-Leu-Tyr-Leu-Val-Cys-Gly-
Glu-Arg-Gly-Phe-Phe-Tyr-Thr-Pro-Glu-Thr-
Glu-Ala-Gly-Pro-Arg-Arg-Gly-Ile-Val-Glu-
Gln-Cys-Cys-His-Ser-Ile-Cys-Ser-Leu-Glu-
Gln-Leu-Gln-Asn-Tyr-Cys-Asn.

In another embodiment, the amino-acid sequence of the single-chain insulin, designated EA8, EA14, RA17, EP, EAGPRR, is provided as SEQ ID NO: 8.

SEQ ID NO: 8
Phe-Val-Asn-Gln-His-Leu-Cys-Gly-Ser-His-
Leu-Val-Glu-Ala-Leu-Tyr-Leu-Val-Cys-Gly-
Glu-Arg-Gly-Phe-Phe-Tyr-Thr-Glu-Pro-Thr-
Glu-Ala-Gly-Pro-Arg-Arg-Gly-Ile-Val-Glu-
Gln-Cys-Cys-Glu-Ser-Ile-Cys-Ser-Leu-Glu-
Gln-Leu-Arg-Asn-Tyr-Cys-Asn.

In another embodiment, the amino-acid sequence of the single-chain insulin, designated HA8, EA14, EB17, PE, EEGPRR, is provided as SEQ ID NO: 9.

SEQ ID NO: 9
Phe-Val-Asn-Gln-His-Leu-Cys-Gly-Ser-His-
Leu-Val-Glu-Ala-Leu-Tyr-Glu-Val-Cys-Gly-
Glu-Arg-Gly-Phe-Phe-Tyr-Thr-Pro-Glu-Thr-
Glu-Glu-Gly-Pro-Arg-Arg-Gly-Ile-Val-Glu-
Gln-Cys-Cys-His-Ser-Ile-Cys-Ser-Leu-Glu-
Gln-Leu-Glu-Asn-Tyr-Cys-Asn.

In still another embodiment, the amino-acid sequence of the single-chain insulin, designated HA8, EA14, FB17, PE, EEGPRR, is provided as SEQ ID NO: 10.

SEQ ID NO: 10
Phe-Val-Asn-Gln-His-Leu-Cys-Gly-Ser-His-
Leu-Val-Glu-Ala-Leu-Tyr-Phe-Val-Cys-Gly-
Glu-Arg-Gly-Phe-Phe-Tyr-Thr-Pro-Glu-Thr-
Glu-Glu-Gly-Pro-Arg-Arg-Gly-Ile-Val-Glu-
Gln-Cys-Cys-His-Ser-Ile-Cys-Ser-Leu-Glu-
Gln-Leu-Glu-Asn-Tyr-Cys-Asn.

In another embodiment, the amino-acid sequence of the single-chain insulin, designated HA8, EA14, FA13, PE, EEGPRR, is provided as SEQ ID NO: 11.

SEQ ID NO: 11
Phe-Val-Asn-Gln-His-Leu-Cys-Gly-Ser-His-
Leu-Val-Glu-Ala-Leu-Tyr-Leu-Val-Cys-Gly-
Glu-Arg-Gly-Phe-Phe-Tyr-Thr-Pro-Glu-Thr-
Glu-Glu-Gly-Pro-Arg-Arg-Gly-Ile-Val-Glu-
Gln-Cys-Cys-His-Ser-Ile-Cys-Ser-Phe-Glu-
Gln-Leu-Glu-Asn-Tyr-Cys-Asn.

In still another embodiment, the amino-acid sequence of the single-chain insulin, designated HA8, EA14, EB16, PE, EEGPRR, is provided as SEQ ID NO: 12.

SEQ ID NO: 12
Phe-Val-Asn-Gln-His-Leu-Cys-Gly-Ser-His-
Leu-Val-Glu-Ala-Leu-Glu-Leu-Val-Cys-Gly-
Glu-Arg-Gly-Phe-Phe-Tyr-Thr-Pro-Glu-Thr-
Glu-Glu-Gly-Pro-Arg-Arg-Gly-Ile-Val-Glu-
Gln-Cys-Cys-His-Ser-Ile-Cys-Ser-Leu-Glu-
Gln-Glu-Glu-Asn-Tyr-Cys-Asn.

In yet another embodiment, the amino-acid sequence of the single-chain insulin, designated HA8, EA14, RA17, PE, EAGPRR, is provided as SEQ ID NO: 13.

SEQ ID NO: 13
Phe-Val-Asn-Gln-His-Leu-Cys-Gly-Ser-His-
Leu-Val-Glu-Ala-Leu-Tyr-Leu-Val-Cys-Gly-
Glu-Arg-Gly-Phe-Phe-Tyr-Thr-Pro-Glu-Thr-
Glu-Ala-Gly-Pro-Arg-Arg-Gly-Ile-Val-Glu-
Gln-Cys-Cys-His-Ser-Ile-Cys-Ser-Leu-Glu-
Gln-Leu-Arg-Asn-Tyr-Cys-Asn.

In still another embodiment, the amino-acid sequence of the single-chain insulin, designated HA8, WA13, EA14, PE, EAGPRR, is provided as SEQ ID NO: 14.

SEQ ID NO: 14
Phe-Val-Asn-Gln-His-Leu-Cys-Gly-Ser-His-
Leu-Val-Glu-Ala-Leu-Tyr-Leu-Val-Cys-Gly-
Glu-Arg-Gly-Phe-Phe-Tyr-Thr-Pro-Glu-Thr-
Glu-Ala-Gly-Pro-Arg-Arg-Gly-Ile-Val-Glu-
Gln-Cys-Cys-His-Ser-Ile-Cys-Ser-Trp-Glu-
Gln-Leu-Gln-Asn-Tyr-Cys-Asn.

In still another embodiment, the amino-acid sequence of the single-chain insulin, designated EA8, EA14, QA17, EP, EAGPRR, is provided as SEQ ID NO: 15.

SEQ ID NO: 15
Phe-Val-Asn-Gln-His-Leu-Cys-Gly-Ser-His-
Leu-Val-Glu-Ala-Leu-Tyr-Leu-Val-Cys-Gly-
Glu-Arg-Gly-Phe-Phe-Tyr-Thr-Glu-Pro-Thr-
Glu-Ala-Gly-Pro-Arg-Arg-Gly-Ile-Val-Glu-
Gln-Cys-Cys-Glu-Ser-Ile-Cys-Ser-Leu-Glu-
Gln-Leu-Gln-Asn-Tyr-Cys-Asn.

In still another embodiment, the amino-acid sequence of the single-chain insulin, designated HA8, EA14, QA17, EP, EAGPRR, is provided as SEQ ID NO: 16.

SEQ ID NO: 16
Phe-Val-Asn-Gln-His-Leu-Cys-Gly-Ser-His-
Leu-Val-Glu-Ala-Leu-Tyr-Leu-Val-Cys-Gly-
Glu-Arg-Gly-Phe-Phe-Tyr-Thr-Glu-Pro-Thr-
Glu-Ala-Gly-Pro-Arg-Arg-Gly-Ile-Val-Glu-
Gln-Cys-Cys-His-Ser-Ile-Cys-Ser-Leu-Glu-
Gln-Leu-Gln-Asn-Tyr-Cys-Asn.

In still another embodiment, the amino-acid sequence of the single-chain insulin, designated HA8, EA14, RA17, EP, EAGPRR, is provided as SEQ ID NO: 17.

SEQ ID NO: 17
Phe-Val-Asn-Gln-His-Leu-Cys-Gly-Ser-His-
Leu-Val-Glu-Ala-Leu-Tyr-Leu-Val-Cys-Gly-
Glu-Arg-Gly-Phe-Phe-Tyr-Thr-Glu-Pro-Thr-
Glu-Ala-Gly-Pro-Arg-Arg-Gly-Ile-Val-Glu-
Gln-Cys-Cys-His-Ser-Ile-Cys-Ser-Leu-Glu-
Gln-Leu-Arg-Asn-Tyr-Cys-Asn.

In still another embodiment, the amino-acid sequence of the single-chain insulin, designated HA8, WA13, EA14, EP, EAGPRR, is provided as SEQ ID NO: 18.

SEQ ID NO: 18
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Glu-Pro-Thr-Glu-Ala-Gly-Pro-Arg-Arg-
Gly-Ile-Val-Glu-Gln-Cys-Cys-His-Ser-Ile-Cys-Ser-
Trp-Glu-Gln-Leu-Glu-Asn-Tyr-Cys-Asn.

In still another embodiment, the amino-acid sequence of the single-chain insulin, designated EA8, LA14, QA17, PE, EAGPRR, is provided as SEQ ID NO: 19.

SEQ ID NO: 19
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Glu-Thr-Glu-Ala-Gly-Pro-Arg-Arg-
Gly-Ile-Val-Glu-Gln-Cys-Cys-Glu-Ser-Ile-Cys-Ser-
Leu-Leu-Gln-Leu-Gln-Asn-Tyr-Cys-Asn.

In yet another embodiment, the amino-acid sequence of the single-chain insulin, designated HA8, CA10, EA14, CB4, HB22, PE, EEGPAH, is provided as SEQ ID NO: 20.

SEQ ID NO: 20
Phe-Val-Asn-Cys-His-Leu-Cys-Gly-Ser-His-Leu-Val-
Glu-Ala-Leu-Tyr-Leu-Val-Cys-Gly-Glu-Cys-Gly-Phe-
Phe-Tyr-Thr-Pro-Glu-Thr-Glu-Glu-Gly-Pro-Ala-His-
Gly-Ile-Val-Glu-Gln-Cys-Cys-His-Ser-Cys-Cys-Ser-
Leu-Glu-Gln-Leu-Glu-Asn-Tyr-Cys-Asn.

In yet another embodiment, the amino-acid sequence of the single-chain insulin, designated HA8, CA10, EA14, CB4, AB22, PE, EEGPAH, is provided as SEQ ID NO: 21.

SEQ ID NO: 21
Phe-Val-Asn-Cys-His-Leu-Cys-Gly-Ser-His-Leu-Val-
Glu-Ala-Leu-Tyr-Leu-Val-Cys-Gly-Glu-Ala-Gly-Phe-
Phe-Tyr-Thr-Pro-Glu-Thr-Glu-Glu-Gly-Pro-Ala-His-
Gly-Ile-Val-Glu-Gln-Cys-Cys-His-Ser-Cys-Cys-Ser-
Leu-Glu-Gln-Leu-Glu-Asn-Tyr-Cys-Asn.

The insulin analogues of the present invention were compared to prior insulin analogues such as insulin lispro (alternatively referred to as “KP” insulin), which contains a B-chain sequence with the substitutions Lys^{B28}, Pro^{B29} as shown in SEQ ID NO: 22.

(lispro)
SEQ ID NO: 22
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Lys-Pro-Thr

The A-chain sequence of insulin lispro would be that of SEQ ID NO: 2 provided above.

Another prior insulin analogue used for comparative purposes would be one designated “DB10” herein, containing an Asp substitution at position B10 as shown in SEQ ID NO: 23:

(DB10)
SEQ ID NO: 23
Phe-Val-Asn-Gln-His-Leu-Cys-Gly-Ser-Asp-Leu-Val-
Glu-Ala-Leu-Tyr-Leu-Val-Cys-Gly-Glu-Arg-Gly-Phe-
Phe-Tyr-Thr-Pro-Lys-Thr

A further variation combines the substitutions of insulin lispro with DB10, as in SEQ ID NO: 24. This analogue is sometimes referred to as “DKP” insulin, for each of the substitutions.

(DKP)
SEQ ID NO: 24
Phe-Val-Asn-Gln-His-Leu-Cys-Gly-Ser-Asp-Leu-Val-
Glu-Ala-Leu-Tyr-Leu-Val-Cys-Gly-Glu-Arg-Gly-Phe-
Phe-Tyr-Thr-Lys-Pro-Thr

In both DB10 and DKP insulin, the A-chain sequence is that of SEQ ID NO: 2 above.

In another embodiment, the amino-acid sequence of the single-chain insulin, designated EA8, EA14, RA17, PE, EAGPRR, is provided as SEQ ID NO: 25.

SEQ ID NO: 25
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Glu-Thr-Glu-Ala-Gly-Pro-Arg-Arg-
Gly-Ile-Val-Glu-Gln-Cys-Cys-Glu-Ser-Ile-Cys-Ser-
Leu-Glu-Gln-Leu-Arg-Asn-Tyr-Cys-Asn.

In yet another embodiment, the amino-acid sequence of the single-chain insulin, designated HA8, EA14, RA17, PE, EEGPRR, is provided as SEQ ID NO: 26.

SEQ ID NO: 26
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Glu-Thr-Glu-Glu-Gly-Pro-Arg-Arg-
Gly-Ile-Val-Glu-Gln-Cys-Cys-His-Ser-Ile-Cys-Ser-
Leu-Glu-Gln-Leu-Arg-Asn-Tyr-Cys-Asn.

In another embodiment, the amino-acid sequence of the single-chain insulin, designated HA8, EA14, NB17, PE, EEGPRR, is provided as SEQ ID NO: 27.

SEQ ID NO: 27
Phe-Val-Asn-Gln-His-Leu-Cys-Gly-Ser-His-Leu-Val-
Glu-Ala-Leu-Tyr-Asn-Val-Cys-Gly-Glu-Arg-Gly-Phe-
Phe-Tyr-Thr-Pro-Glu-Thr-Glu-Glu-Gly-Pro-Arg-Arg-
Gly-Ile-Val-Glu-Gln-Cys-Cys-His-Ser-Ile-Cys-Ser-
Leu-Glu-Gln-Leu-Glu-Asn-Tyr-Cys-Asn.

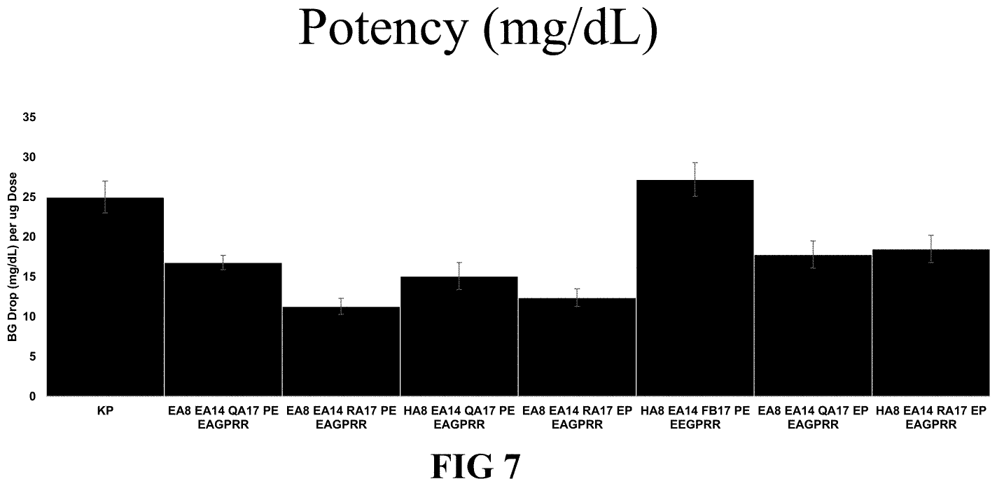

The potency of several embodiments of the present insulin analogue was evaluated in male diabetic Lewis rats that were rendered diabetic by treatment with streptozotocin (STZ). Experimental analogs, insulin lispro (KP), and diluent only control solutions were injected subcutaneously, and the resulting changes in blood glucose (BG) were monitored by serial measurements using a clinical glucometer. Dose response was calculated as maximal BG drop in excess of the diluent control and plotted. Langmuir isotherm was fitted using iterative weighting to calculate the best-fit dose response curve and EC50 determined as the interpolated dose required to achieve a drop half way between fitted maximal and minimal drops. The resulting values are provided in Table 1 below and in FIG. 7.

TABLE 1

| | BG Drop (mg/dL) |
|---|---|
| Insulin analogue | per μg Dose |
| KP (SEQ ID NOS: 2 and 22) | 25 |
| EA8 EA14 QA17 PE EAGPRR (SEQ ID NO: 5) | 16.8 |
| EA8 EA14 RA17 PE EAGPRR (SEQ ID NO: 6) | 11.3 |
| HA8 EA14 QA17 PE EAGPRR (SEQ ID NO: 7) | 15.1 |
| EA8 EA14 RA17 EP EAGPRR (SEQ ID NO: 8) | 12.4 |
| HA8 EA14 FB17 PE EEGPRR (SEQ ID NO: 10) | 27.2 |
| HA8 EA14 NB17 PE EEGPRR (SEQ ID NO: 27) | 15.2 |
| EA8 EA14 QA17 EP EAGPRR (SEQ ID NO: 15) | 17.8 |
| HA8 EA14 RA17 EP EAGPRR (SEQ ID NO: 17) | 18.5 |

Representative analogues of the present invention were found to retain a substantial proportion of the biological activity of insulin lispro. The analogues tested display at least 45 percent of the potency of insulin lispro. In most instances, the analogues have more than half the potency of insulin lispro, and one analogue has greater potency than insulin lispro.

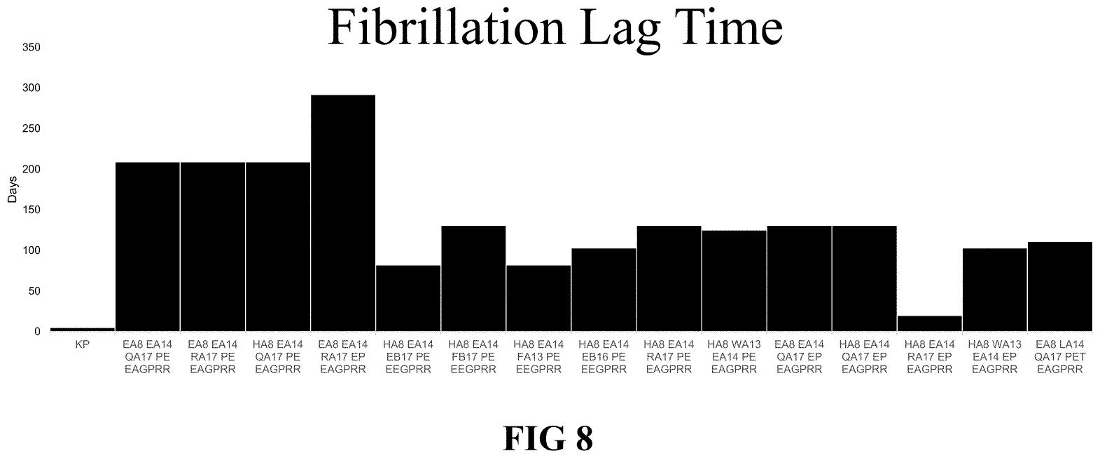

Resistance to fibrillation was determined by gentle agitation of samples formulated to a final concentration of U10 using Phosphate Buffered Saline (PBS), pH 7.4. 1 uM of Thioflavin T (ThT) was added to each solution and 150 uL was added to each well. The plate was incubated at 40° C. with a constant linear shake of 1000 cpms. Sampling was performed daily with excitation/emission wavelengths of 440/480 nm. Results are provided in Table 2 and FIG. 8.

TABLE 2

| Analogue | U10 Fibril Days |
|---|---|
| KP (SEQ ID NOS: 2 and 22) | 5 |
| EA8 EA14 QA17 PE EAGPRR (SEQ ID NO: 5) | 209 |
| EA8 EA14 RA17 PE EAGPRR (SEQ ID NO: 6) | 209 |
| HA8 EA14 QA17 PE EAGPRR (SEQ ID NO: 7) | 209 |
| EA8 EA14 RA17 EP EAGPRR (SEQ ID NO: 8) | 292 |
| HA8 EA14 EB17 PE EEGPRR (SEQ ID NO: 9) | 82 |
| HA8 EA14 FB17 PE EEGPRR (SEQ ID NO: 10) | 131 |
| HA8 EA14 FA13 PE EEGPRR (SEQ ID NO: 11) | 82 |
| HA8 EA14 EB16 PE EEGPRR (SEQ ID NO: 12) | 103 |
| HA8 EA14 RA17 PE EAGPRR (SEQ ID NO: 13) | 131 |
| HA8 WA13 EA14 PE EAGPRR (SEQ ID NO: 14) | 125 |
| EA8 EA14 QA17 EP EAGPRR (SEQ ID NO: 15) | 131 |
| HA8 EA14 QA17 EP EAGPRR (SEQ ID NO: 16) | 131 |
| HA8 EA14 RA17 EP EAGPRR (SEQ ID NO: 17) | 20 |
| HA8 WA13 EA14 EP EAGPRR (SEQ ID NO: 18) | 103 |
| EA8 LA14 QA17 PE EAGPRR (SEQ ID NO: 19) | 111 |

The data provided herein demonstrates that the analogues tested provide at least a four-fold longer fibrillation time than insulin lispro. In most instances, the analogues exhibit more than a ten-fold or even twenty-fold longer fibrillation time. In some cases the analogues displayed more than a forty-fold longer fibrillation time.

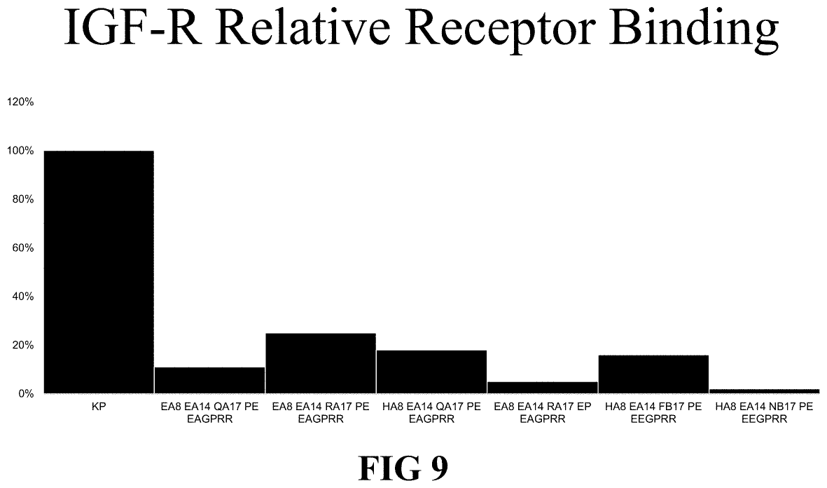

Cross-reactivity of the single-chain insulin analogues with IGF-1R was studied using a FLAG epitope-tagged holoreceptor to human type 1 insulin-like growth factor receptor (hIGFR) bound to 96 well plates coated with anti-FLAG monoclonal antibody. Relative affinity is defined as the ratio of dissociation constants as determined by competitive displacement of bound ¹²⁵I labeled human IGF-I. Dissociation constants (K_{d}) were determined by fitting to a mathematic model as described by Whittaker and Whittaker (2005. J. Biol. Chem. 280, 20932-20936); the model employed non-linear regression with the assumption of heterologous competition (Wang, 1995, FEBS Lett. 360, 111-114). This is used as a marker for mitogenic potential, where less IGF-R affinity is interpreted as lower mitogenic potential. Results are provided in Table 3 and FIG. 9.

TABLE 3

| | IGF1-R (Rel. Aff.) |
|---|---|
| Insulin Analogue | ≤ HI/KP |
| KP (SEQ ID NOS: 2 and 22) | 100% |
| EA8 EA14 QA17 PE EAGPRR (SEQ | 11% |
| ID NO: 5) | |
| EA8 EA14 RA17 PE EAGPRR (SEQ | 25% |
| ID NO: 6) | |
| HA8 EA14 QA17 PE EAGPRR | 18% |
| (SEQ ID NO: 7) | |
| EA8 EA14 RA17 EP EAGPRR | 5% |
| (SEQ ID NO: 8) | |
| HA8 EA14 FB17 PE EEGPRR | 16% |
| (SEQ ID NO: 10) | |
| HA8 EA14 NB17 PE EEGPRR (SEQ | 2% |
| ID NO: 27) | |

The insulin analogues tested each displayed a reduced affinity for IGF-1R compared to insulin lispro. Specifically, the analogues displayed 25 percent or less affinity than insulin lispro. In most cases, the insulin analogues displayed less than 20 percent affinity for IGF-1R compared to insulin lispro. In some cases, the analogues displayed less than 10 percent affinity for IGF-1R compared to insulin lispro.

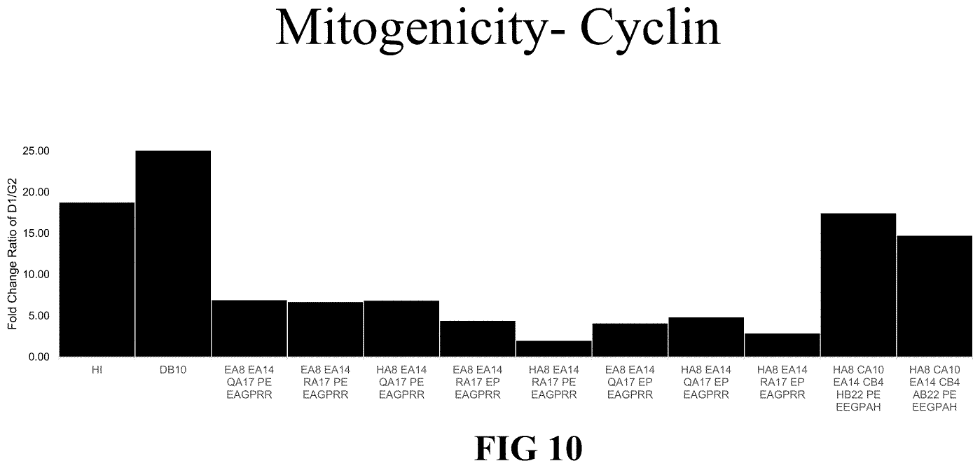

To confirm the reduced mitogenicity of the present single-chain insulin analogues, RT-qPCR assays, monitoring the transcription responses of mitogenicity probes stimulated by treatment of different insulin analogs, were performed. Expression regulation of two cyclins served as major probes: Cyclin D1 is up-regulated whereas cyclin G2 is down-regulated correlated to the active cell division cycle (proliferation, which is generally correlated to mitogenicity). A ratio of D1/G2 transcription levels gives a picture of the mitogenic potential of a compound; a higher ratio means more mitogenic potential. In this assay, a rat myoblast cell line (L6) with high-expression of insulin receptor (IR) served as the cell model. Results are provided in Table 4 and FIG. 10.

TABLE 4

| | | | Cyclin |
|---|---|---|---|
| Insulin/Insulin analogue | D1 | G2 | D1/G2 |
| HI (SEQ ID NOS: 2 and 3) | 5.45 | 0.29 | 18.79 |
| DB10 (SEQ ID NOS: 2 and 23) | 18.04 | 0.13 | 138.77 |
| EA8 EA14 QA17 PE EAGPRR | 3.9 | 0.56 | 6.96 |
| (SEQ ID NO: 5) | | | |
| EA8 EA14 RA17 PE EAGPRR | 4.5 | 0.67 | 6.72 |
| (SEQ ID NO: 6) | | | |
| HA8 EA14 QA17 PE EAGPRR | 3.1 | 0.45 | 6.89 |
| (SEQ ID NO: 7) | | | |
| EA8 EA14 RA17 EP EAGPRR | 2.8 | 0.63 | 4.44 |
| (SEQ ID NO: 8) | | | |
| HA8 EA14 RA17 PE EAGPRR | 1.72 | 0.85 | 2.02 |
| (SEQ ID NO: 13) | | | |
| EA8 EA14 QA17 EP EAGPRR | 2.02 | 0.49 | 4.12 |
| (SEQ ID NO: 15) | | | |
| HA8 EA14 QA17 EP EAGPRR | 1.46 | 0.3 | 4.87 |
| (SEQ ID NO: 16) | | | |
| HA8 EA14 RA17 EP EAGPRR | 1.49 | 0.51 | 2.92 |
| (SEQ ID NO: 17) | | | |
| HA8 CA10 EA14 CB4 HB22 PE | 3.5 | 0.2 | 17.50 |
| EEGPAH (SEQ ID NO: 20) | | | |
| HA8 CA10 EA14 CB4 AB22 PE | 3.25 | 0.22 | 14.77 |
| EEGPAH (SEQ ID NO: 21) | | | |

The data indicate that each of the single-chain insulin analogues of the present invention have a reduced ratio of Cyclin D1/G2 compared to both human insulin (HI) and DB10 insulin. In most instances the Cyclin D1/G2 ratio is less than half that of human insulin.

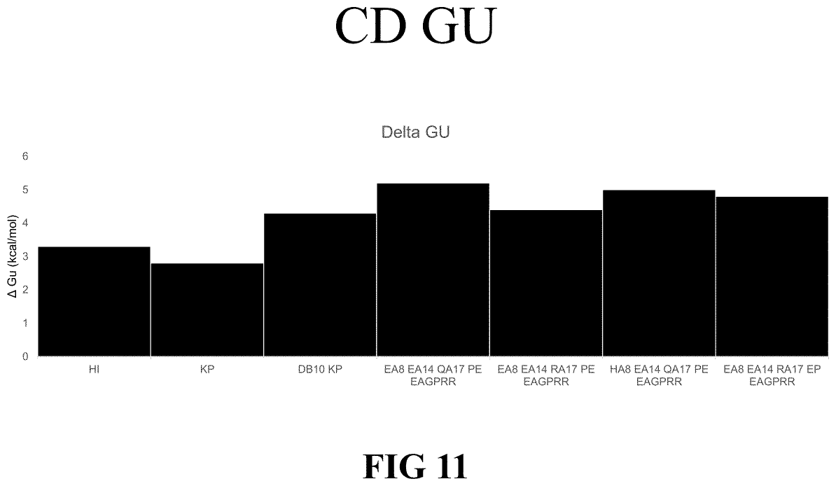

Thermodynamic stability of the present single-chain insulin analogues was evaluated at 25° C. and pH 7.4 by circular dichroism (CD)-monitored guanidine denaturation. The free energy of unfolding (ΔGᵤ) of each of the single-chain insulin analogues tested was greater than each of human insulin (HI), insulin lispro (KP) and even DB10 KP (DKP) insulin, as shown in Table 5 and FIG. 11. This increase in free energy predicts enhanced chemical stability.

TABLE 5

| | ΔGu |
|---|---|
| Insulin/Insulin analogue | (kcal/mol) |
| HI (SEQ ID NOS: 2 and 3) | 3.3 |
| KP (SEQ ID NOS: 2 and 22) | 2.8 |
| DB10 KP (SEQ ID NO: 2 and 24) | 4.3 |
| EA8 EA14 QA17 PE EAGPRR | 5.2 |
| (SEQ ID NO: 5) | |
| EA8 EA14 RA17 PE EAGPRR | 4.4 |
| (SEQ ID NO: 6) | |
| HA8 EA14 QA17 PE EAGPRR | 5 |
| (SEQ ID NO: 7) | |
| EA8 EA14 RA17 EP EAGPRR | 4.8 |
| (SEQ ID NO: 8) | |

A method for treating a patient with diabetes mellitus comprises administering a single-chain insulin analogue as described herein. It is another aspect of the present invention that the single-chain insulin analogues may be prepared either in yeast (Pichia pastoris) or subject to total chemical synthesis by native fragment ligation. We further envision the analogues of the present invention providing a method for the treatment of diabetes mellitus or the metabolic syndrome. The route of delivery of the insulin analogue is by subcutaneous injection through the use of a needle and syringe or pen device.

A single-chain insulin analogue of the present invention may also contain other modifications, such as a halogen atom at positions B24, B25, or B26 as described more fully in co-pending U.S. Pat. No. 8,921,313, the disclosure of which is incorporated by reference herein. An insulin analogue of the present invention may also contain a foreshortened B-chain due to deletion of residues B1-B3 as described more fully in co-pending U.S. Pat. No. 9,725,493.

A pharmaceutical composition may comprise such insulin analogues and which may optionally include zinc. Zinc ions may be included at varying zinc ion:protein ratios, ranging from 2.2 zinc atoms per insulin analogue hexamer to 3 zinc atoms per insulin analogue hexamer. The pH of the formulation is in the range pH 6.8-8.0. In such a formulation, the concentration of the insulin analogue would typically be between about 0.6-5.0 mM; concentrations up to 5 mM may be used in vial or pen; the more concentrated formulations (U-200 or higher) may be of particular benefit in patients with marked insulin resistance. Excipients may include glycerol, glycine, arginine, Tris, other buffers and salts, and anti-microbial preservatives such as phenol and meta-cresol; the latter preservatives are known to enhance the stability of the insulin hexamer. Single-chain insulin analogues may be formulated in the presence of zinc ions or in their absence. Such a pharmaceutical composition as described above may be used to treat a patient having diabetes mellitus or other medical condition by administering a physiologically effective amount of the composition to the patient.

Based upon the foregoing disclosure, it should now be apparent that the single-chain insulin analogues provided will carry out the objects set forth hereinabove. Namely, these insulin analogues exhibit both accelerated absorption into the blood stream from a subcutaneous depot (“fast on”) and foreshortened duration of signaling (“fast off) while maintaining at least a fraction of the biological activity of wild-type insulin. It is, therefore, to be understood that any variations evident fall within the scope of the claimed invention and thus, the selection of specific component elements can be determined without departing from the spirit of the invention herein disclosed and described.

The following literature is cited to demonstrate that the testing and assay methods described herein would be understood by one of ordinary skill in the art.
Glendorf, T., Knudsen, L., Stidsen, C. E., Hansen, B. F., Hegelund, A. C., Sorensen, A. R., Nishimura, E., & Kjeldsen, T. 2012. Systematic evaluation of the metabolic to mitogenic potency ratio for B10-substituted insulin analogues. PLoS One 7(2), e29198.
Hohsaka, T., & Sisido, M. 2012. Incorporation of non-natural amino acids into proteins. Curr. Opin. Chem. Biol. 6, 809-15.
Hua, Q. X., Nakagawa, S. H., Jia, W., Huang, K., Phillips, N. B., Hu, S. & Weiss, M. A. (2008) Design of an active ultrastable single-chain insulin analog: synthesis, structure, and therapeutic implications. J. Biol. Chem. 283, 14703-14716.
Kristensen, C., Andersen, A. S., Hach, M., Wiberg, F. C., Schïffer, L., & Kjeldsen, T. 1995. A single-chain insulin-like growth factor I/insulin hybrid binds with high affinity to the insulin receptor. Biochem. J. 305, 981-6.
Lee, H. C., Kim, S. J., Kim, K. S., Shin, H. C., & Yoon, J. W. 2000. Remission in models of type 1 diabetes by gene therapy using a single-chain insulin analogue. Nature 408, 483-8. Retraction in: Lee H C, Kim K S, Shin H C. 2009. Nature 458, 600.
Phillips, N. B., Whittaker, J., Ismail-Beigi, F., & Weiss, M. A. (2012) Insulin fibrillation and protein design: topological resistance of single-chain analogues to thermal degradation with application to a pump reservoir. J. Diabetes Sci. Technol. 6, 277-288.
Sciacca, L., Cassarino, M. F., Genua, M., Pandini, G., Le Moli, R., Squatrito, S., & Vigneri, R. 2010. Insulin analogues differently activate insulin receptor isoforms and post-receptor signalling. Diabetologia 53, 1743-53.
Wang, Z. X. 1995. An exact mathematical expression for describing competitive biding of two different ligands to a protein molecule FEBS Lett. 360: 111-114.
Whittaker, J., and Whittaker, L. 2005. Characterization of the functional insulin binding epitopes of the full-length insulin receptor. J. Biol. Chem. 280: 20932-20936.

What is claimed is:

1. A single-chain insulin analogue comprising an insulin B-chain polypeptide sequence connected by a C-domain connecting polypeptide sequence to an insulin A-chain polypeptide sequence, wherein the C-domain connecting polypeptide sequence is selected from the group consisting of the sequence Glu-Glu-Gly-Pro-Ala-His and the sequence Glu-Xaa-Gly-Pro-Arg-Arg where Xaa is Glu or Ala.

2. The single-chain insulin analogue of claim 1, additionally comprising Glu or His substitutions at the position corresponding to A8 of human insulin, a Glu substitution at the position corresponding to A14 of human insulin, or a combination thereof.

3. The single-chain insulin analogue of claim 2, additionally comprising either a Pro or Glu at the positions corresponding to B28 and B29 of wild-type insulin.

4. The single-chain insulin analogue of claim 3, additionally comprising a Phe or Trp substitution at the position corresponding to A13 of wild type insulin, a Gln, Arg, Phe, or Glu at the position corresponding to A17 of wild type insulin, or a combination thereof.

5. The single-chain insulin analogue of claim 4, wherein the C-domain connecting polypeptide sequence is Glu-Ala-Gly-Pro-Arg-Arg.

6. The single-chain insulin analogue of claim 5, comprising any one of SEQ ID NOS: 5-8 and 13-19.

7. The single-chain insulin analogue of claim 6, comprising an Arg substitution at the position corresponding to A17 of wild type insulin.

8. The single-chain insulin analogue of claim 7, comprising SEQ ID NO: 8.

9. The single-chain insulin analogue of claim 5, comprising any one of SEQ ID NOS: 13 and 17.

10. The single-chain insulin analogue of claim 3, wherein the C-domain connecting polypeptide sequence is Glu-Glu-Gly-Pro-Arg-Arg.

11. The single-chain insulin analogue of claim 5, comprising any one of SEQ ID NOS: 9-12.

12. The single-chain insulin analogue of claim 1, additionally comprising a Glu substitution at the position corresponding to B16 of wild type insulin may be present.

13. The single-chain insulin analogue of claim 1, additionally comprising a Cys substitution at the positions corresponding to A10 and/or B4 of wild-type insulin.

14. The single-chain insulin analogue of claim 1, additionally comprising a His or Ala substitution at the position corresponding to B22 of wild-type insulin

15. (canceled)

16. (canceled)

17. (canceled)

18. A method of lowering the blood sugar of a patient, the method comprising administering a pharmaceutically effective amount of an insulin analogue to the patient, wherein the insulin analogue comprises an insulin B-chain polypeptide sequence connected by a C-domain connecting polypeptide sequence to an insulin A-chain polypeptide sequence, wherein the C-domain connecting polypeptide sequence is selected from the group consisting of the sequence Glu-Glu-Gly-Pro-Ala-His and the sequence Glu-Xaa-Gly-Pro-Arg-Arg where Xaa is Glu or Ala.

19. The method of lowering the blood sugar of a patient according to claim 18, wherein the insulin analogue additionally comprises Glu or His substitutions at the position corresponding to A8 of human insulin, a Glu substitution at the position corresponding to A14 of human insulin, or a combination thereof.

20. The method of lowering the blood sugar of a patient according to claim 18, wherein the C-domain connecting polypeptide sequence is the sequence Glu-Glu-Gly-Pro-Ala-His.

21. The method of lowering the blood sugar of a patient according to claim 18, wherein the insulin analogue additionally comprises a Phe or Trp substitution at the position corresponding to A13 of wild type insulin, a Gln, Arg, Phe, or Glu at the position corresponding to A17 of wild type insulin, or a combination thereof.

22. The method of lowering the blood sugar of a patient according to claim 20, wherein the insulin analogue additionally comprises a Phe or Trp substitution at the position corresponding to A13 of wild type insulin, a Gln, Arg, Phe, or Glu at the position corresponding to A17 of wild type insulin, or a combination thereof.

23. The single-chain insulin analogue of claim 1, wherein the C-domain connecting polypeptide sequence is Glu-Glu-Gly-Pro-Ala-His.
